(19) United States
(12) Patent Application Publication
(10) Pub. No.: US 2006/0031022 A1
(43) Pub. Date: Feb. 9, 2006

(54) Method of planning and performing stability studies

(76) Inventor: Philip Hougaard, Bagsvaerd (DK)

Correspondence Address: NOVO NORDISK, INC.;PATENT DEPARTMENT, 100 COLLEGE ROAD WEST, PRINCETON, NJ, 08540, US

(21) Appl. No.: 11/243,014

(22) Filed: Oct. 4, 2005

Related U.S. Application Data
(63) Continuation of application No. 10/006,601, filed on Dec. 4, 2001.

(51) Int. Cl. G06F 19/00; A61B 5/00; G01N 33/48; G01N 33/50

(52) U.S. Cl. 702/19; 705/3

(57) ABSTRACT

The present invention relates to a method and system for planning a stability study of a pharmaceutical composition. According to the current invention a new statistical principle for designing studies is provided. It addresses directly that the aim of the stability study is to derive more precise and efficient specification limits. The method involves making estimates of the needs that might be encountered and in that way determine whether a given stability study model can provide the precision necessary to derive appropriate shelf-life specifications. The approach is based on utilizing normal distribution calculations of the obtainable specifications in Allen's formula. The terms that are estimated include the degradation rates such that in the estimated model, the specifications arrived at have at least a 90% chance of being better than projected by other methods. In addition the standard evaluation of the uncertainty of the slope is performed. Data at accelerated temperatures are other conditions may also be included to increase precision.

### FIELD OF THE INVENTION

The present invention relates to methods for planning stability studies for pharmaceutical compositions. More specifically, the current invention provides a method to enhance existing pharmaceutical stability study planning methodologies and thereby improve upon the precision necessary to derive useful estimations of pharmaceutical shelf-life as well as decide on the size of any stability study later conducted on a pharmaceutical composition.

### BACKGROUND OF THE INVENTION

The present invention is directed to a method for planning, evaluating, and improving the precision of pharmaceutical stability studies, thereby enhancing the precision of pharmaceutical preparation and providing for evaluation of specifications of the drug. Specifically, the method of the invention can be used to determine the size of the stability study to obtain a specified precision for the results.

The Food and Drug Administration requires pharmaceutical companies to establish a shelf-life for all new drug products through the stability analysis of a given pharmaceutical composition. This is done to ensure the quality of the drug taken by an individual is within established levels. (PHARMACEUTICAL DOSAGE FORMS AND DRUG DELIVERY SYSTEMS, Ansel, Popovich, and Allen, (6th edition)). Typically, this is done through using simple linear-regression models and thereafter interpreting confidence and prediction intervals.

Pharmaceutical companies estimate the shelf-life, and therefore the expiration date, of a drug to determine the amount of time the drug is at acceptable potency and color, levels in a particular formulation and/or packaging configuration. The acceptable levels are set by the pharmaceutical company or the Food and Drug Administration. The process in which the shelf-life is determined is called a stability analysis, and must be established through a stability study. The shelf-life of a drug is generally defined as the length of time a drug can stay on the shelf without degrading to unacceptable levels of chemical potency or pharmaceutical utility.

A determination of pharmaceutical stability is based on the testing of randomly selected samples from a particular batch of the drug in question at particular time points and/or temperature points after production for analysis of chemical, physical, or microbiologic degradation. (Connor, K. A., et al. in CHEMICAL STABILITY OF PHARMACEUTICALS—A HANDBOOK FOR PHARMACIST, pp. 1-37 (John Wiley & Sons, 1986, New York)). With this data, standard regression analysis models can be used to provide an estimate of potency over different time intervals. Thereafter confidence and prediction intervals for the pharmaceutical composition of interest are plotted, yielding shelf-life estimates with a high level of confidence. The shelf-life is the time interval in which the 95% confidence interval band intersects the lines corresponding to the requested limits on the potency.

United States regulations concerning the stability studies needed for the estimation of the shelf-life of a pharmaceutical formulation typically follow the International Committee of Harmonization (I.C.H.) guidelines. In June 1998, the FDA released a draft of the I.C.H. guidelines designed to help drug manufacturers through required stability studies. The FDA's draft covered stability studies for new drug applications (NDA's), abbreviated NDA's, and investigational NDA's. These guidelines are also followed by Japan and most of Europe. Different methodologies can be used to determine pharmaceutical stability. Examples include a kinetic extrapolation method developed according to the procedure described by I.C.H. guidelines; another is based on a thermal extrapolation and linear-regression according to the Arrhenius Theory.

A more advanced approach to evaluate specifications was described by Allen (Allen, Paul V. et al.: Determination of Release Limits: A General Methodology, PHARM. RES. 8:1210 (1991), incorporated herein by reference), consisting of evaluating a minimum necessary difference between the release and shelf-life limits, accounting not only for the uncertainty in the stability study to be carried out, but also for the uncertainty on the measurements taken for releasing the batch.

For pharmaceutical products undergoing clinical testing, a stability study is normally conducted to calculate a shelf-life, also known as the expiratory dating period. A comparison of several methods for computing the expiration-dating period, the shelf-life, is often explored using real datasets. All methods are based upon a linear-regression procedure. The method for the traditional NDA three batch sample is to consider batches as fixed and take the batch with the shortest expiration-dating period. When marketing batches become available there may be many more than three batches and this fixed effects methodology may not give realistic answers. Fixed effects methods include calculations using fixed effects regression models with and without common error, and common slope. Random coefficients models were also fit with slopes and intercepts independent and with an unstructured covariance matrix. Prediction limits, confidence limits and tolerance limits were calculated with these random effects models and compared to the fixed effects models.

The shelf-life and stability of all pharmaceutical agents is of great importance. Through the use of chemical kinetics one can predict the rate and course of drug degradation. More efficient models and methods of conducting such studies can save drug designers and manufacturers substantial amounts of time and money during the large-scale production of individual pharmaceutical compositions by contributing precision to shelf-life estimates as well as insuring improved efficacy of pharmaceutical compositions prepared with these methods. Moreover, stability studies often take a minimum of six months to perform, even with accelerated testing, and during that period the drugs cannot be marketed. Any delay, the cause of which can range from an improperly performed test to the discovery that a particular composition or material fails to preserve a certain drug, may affect both the production and commercial availability of a drug.

Stability studies are often designed to conform to the precedent set by previous studies, which may not provide optimal results. The study may end up being either too small so that it is not possible to guarantee that satisfactory specifications can be met, thus resulting in either a shortening of the shelf-life period or a delay of the filing of the drug. Or, a study may be too large, which is a waste of resources, including not only money, but also the drug product that may be sparsely available at that time. Furthermore it can create bottlenecks in the laboratories leading to additional delays in commercialization. In both cases there is a high financial impact. It is a great advantage to design the stability studies according to statistical principles so that the planning can account for the precision of the measurement methods and for the time pressure in the drug development phase.

Known statistical principles for designing studies are the power principle and the standard error principle. The power principle is very widely applied in clinical studies of drugs. (Chow, S. and Liu, J. (1995). STATISTICAL DESIGN AND ANALYSIS IN PHARMACEUTICAL SCIENCE: VALIDATION, PROCESS CONTROLS, AND STABILITY, pp. 5-21, 41-56 (New York: Marcel Dekker, Inc.)). It is based on the study of a statistical hypothesis. Typically this hypothesis is that the drug under study gives the same results as placebo and the study is then designed so that there is a high probability that the drug is better than placebo. This is true if the true difference has a specified relevant size. However, the power principle is not relevant to stability studies because there is no natural hypothesis to consider. The standard error principle appears to be more relevant; it requires that the standard error on the degradation rate should satisfy some chosen requirements. Thus, the problem with the standard error principle, used in the prior art, is that it is very difficult to suggest a relevant limit in practice, making stability studies generated in this way inefficient.

Accordingly, a need exists for improved stability study planning methodologies in the production and testing of pharmaceutical compositions, particularly those utilizing statistical principles.

### SUMMARY OF THE INVENTION

The present invention encompasses improved methods of planning pharmaceutical stability studies and carrying out more efficiently the preparation of pharmaceutical preparations based on those studies.

The method provides a standard approach for choosing the size of long-term drug product stability studies; particularly for NDA stability studies. The approach is aimed at setting specifications, and specifically at finding the difference between release and shelf-life limits by means of Allen's formula. To do so, it must account for the expected degradation and the intermediate precision as well as study specific parameters. The list of parameters includes: the number of batches of a target pharmaceutical prepared; the number of samples at the various time points, and the length of the study at the time of setting the specifications.

Specifically, the current invention provides for a method for planning a stability study of a pharmaceutical composition. The method is comprised of the following steps including: selecting a value for a release limit variable for a given specification test; selecting a desired length of the shelf-life of said pharmaceutical composition; selecting a time at which an analysis of the data for said stability study will be performed in order to set specifications; selecting time points at which one or more measurements of one or more predetermined pharmaceutical test variables can be performed; selecting a number of measurements of said predetermined test variables that will be performed at each of said time points; selecting a value for the expected degradation rate of said pharmaceutical composition over time; selecting a value for the intermediate precision of said measurements; and finally selecting a probability level regarding the level of certainty of the outcome of said stability study.

It should be noted that there is no particular order established with regard to the steps recited above in which a value is selected. According to the current invention, the values can be selected and input in any order into Allen's formula. This also allows a user to alter the values to evaluate the benefits of various parameters before the initiation of a stability study. Once the above steps are completed, the method of the instant invention will allow the shelf-life specification limits of a test or target pharmaceutical composition to be calculated based upon the variables selected in the steps mentioned above.

Moreover, the method of the current invention also provides for optimizing the variables selected in the steps mentioned above by changing one or more of the variables and recalculating the shelf-life specifications as necessary utilizing Allen's Formula. The specification test limits provided by the current invention may also be re-calculated by substituting in actual data obtained during a stability study for one or more of the variables mentioned above. It is desirable that the variables selected and the method of the current invention are followed such that the confidence levels regarding the level of certainty of the shelf-life specifications arrived at are at least 90%, and preferably at 95%.

It is also important to point out that the value selected for the expected degradation rate may be based on previous long-term stability studies. The computed degradation rate may also be based on previous long-term stability studies of a target pharmaceutical composition in an alternate formulation or in a study accelerated by increased temperature. Accelerated stability results reached in this way may be corrected by the Arrhenius formula.

In an additional embodiment of the current invention the value selected for the intermediate precision of the analysis of the target pharmaceutical composition may be determined from previous-long-term stability studies of the same or similar pharmaceutical compositions.

Also according to the instant invention the time points for measurement of the variables mentioned above may be at any time, preferably however these time points are at 0, 3, 6, 9, and 12 months after start of the stability study of a target pharmaceutical composition.

Other features and advantages of this invention will become apparent in the following detailed description of preferred embodiments of this invention, taken with reference to the accompanying drawings.

### BRIEF DESCRIPTION OF THE DRAWINGS

FIG. 1 shows an exemplary specification limit evaluation for an assay.

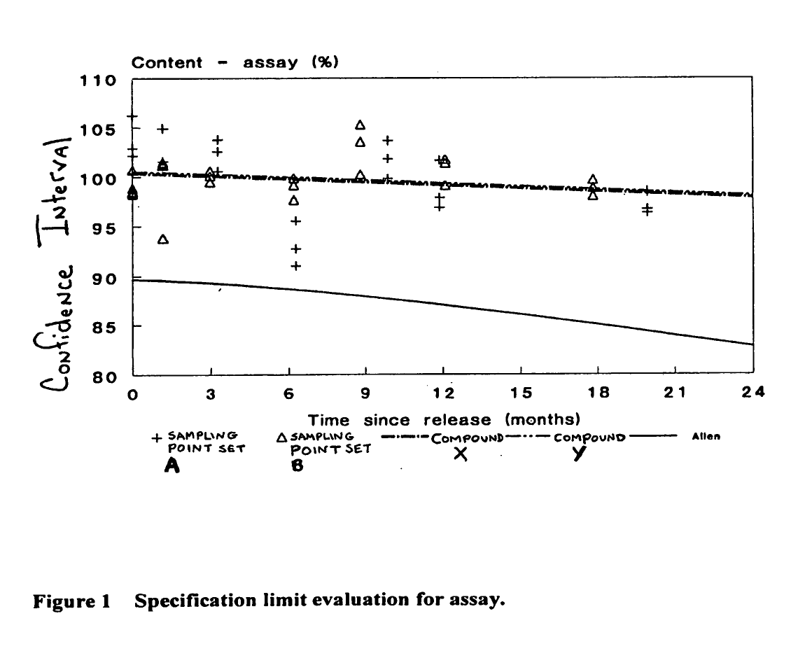

### DESCRIPTION OF THE PREFERRED EMBODIMENT

The following abbreviations have designated meanings in the specification:

Abbreviation Key:
SSL shelf-life limit,
RL release limit,
Δ expected degradation rate
s intermediate precision (determined with df degrees of freedom)
α the probability level of the specifications, typically chosen as 0.95, but for degradation products if may be chosen higher, for example 0.99.
D a non-random factor that depends only on the design, that is, the times the measurements of the various batches are taken in the stability study. More precisely, the standard error of Δ is D s.
T Desired length of Shelf-Life.
k the number of determinations at release (made on different days)
df degrees of freedom for the total variation—that is for the variance term under the square root sign.
S_{Δ} standard error on Δ
ΔE activation energy
R the gas constant
HPLC High Pressure Liquid Chromatography
NDA New Drug Application
RSD Relative Standard Deviations
RE Relative Efficiency

Explanation of Terms:

Accelerated testing. Studies designed to increase the rate of chemical degradation or physical change of a drug substance or drug product by using exaggerated storage conditions as part of the formal studies. These data, in addition to long term stability studies, may also be used to assess longer term chemical effects under non-accelerated conditions and to evaluate the impact of short term excursions outside the label storage conditions such as might occur during shipping. Results from accelerated testing studies are not always predictive of physical changes.

Bracketing. The design of a stability schedule so that at any time point only the samples at the extremes, for example of container size and/or dosage strengths, are tested. The design assumes that the stability of the intermediate condition samples is represented by those at the extremes. Where a range of dosage strengths is to be tested, bracketing designs may be particularly applicable if the strengths are very closely related in composition (e.g., for a tablet range made with different compression weights of a similar basic granulation, or a capsule range made by filling different plug fill weights of the same basic composition into different size capsule shells). Where a range of sizes of immediate containers are to be evaluated, bracketing designs may be applicable if the material of composition of the container and the type of closure are the same throughout the range.

Climatic zones. The concept of dividing the world into four zones based on defining the prevalent annual climatic conditions.

Commitment batches. Production batches of a drug substance or drug product for which the stability studies will be initiated or completed post approval through a commitment made in the Registration Application.

Dosage form. A pharmaceutical product type, for example tablet, capsule, solution, cream etc. that contains a drug substance generally, but not necessarily, in association with excipients.

Drug product. The dosage form in the final immediate packaging intended for marketing.

Drug substance. The unformulated drug substance, which may be subsequently formulated with excipients to produce the drug product.

Excipient. Anything other than the drug substance in the dosage form.
Expiration date. The date placed on the container/labels of a drug product designating the time during which a batch of the product is expected to remain within the approved shelf-life specification if stored under defined conditions, and after which it must not be used.

Formal stability studies. Long term, accelerated and intermediate studies undertaken on primary and/or commitment batches according to a prescribed stability, protocol to establish or confirm the re-test period of a drug substance or the shelf-life of a drug product.

The present invention relates to a system for an improved method for planning, conducting and improving the precision of pharmaceutical stability studies. In this approach existing data is analyzed through the use of mixed models for normally distributed data. (Chen, James J. et al.: Estimation of the Shelf-Life of Drugs with Mixed Effects Models, J. BIOPHARMACEUTICAL STATISTICS, 5(1):131-40 (1995)). Data at accelerated temperatures may be included in a non-linear-regression mixed model based on the Arrhenius equation.

The approach is based on utilizing normal distribution calculations of the obtainable specifications in Allen's formula. In this sense the obtainable terms refer to the allowance for stability study uncertainty in the degradation rates so that the specifications have a 95% chance of being better than those projected by other methods and calculated at the initial planning of the stability studies.

In stability studies of a drug product, a number of samples (that is, vials or Penfill® for insulin; tablets for many other drugs) for a period of time at specified storage conditions. Such a study always includes several batches to ensure that the production process is robust. At various time points, some of these are pulled for analysis of selected test parameters, for example, assay, degradation products, preservatives or other physical or chemical parameters.

I. Analysis of Existing Data According to the Current Invention

Specifications According to Allen's Formula

For planning stability studies the present invention utilizes a formulation that is slightly different from the standard formulation described by Allen. It assumes that the intermediate precision is the same in the stability studies and in the future batches, whereas the standard formulation allows for different values. So the relation imposed is s_{Δ}=D s. The advantage of this is that s can be moved outside of the square root sign. This makes it clearer that there are just two random terms to be determined in the stability study, Δ and s. Furthermore, it is easier to keep track of the degrees of freedom.

In this context, Allen's formula looks like
SLL=RL+T+ts(1/k+D²T²),
where the terms are the following:
SLL shelf-life limit,
RL the release limit,
Δ expected degradation rate
s intermediate precision (determined with df degrees of freedom)
t t-fractile probability, with degrees of freedom df
α the probability level of the specifications, typically chosen as 0.95, but for degradation products it may be chosen higher, such as, for example, 0.99
D a non-random factor that depends only on the design, that is, the time points of measurements of the various batches in the stability study. More precisely, the standard error of Δ is D s
T the length of shelf-life
k the number of determinations at release (made on different days)

As such, the formula is presented for a single product in a single type of package and under a single storage condition. However, under an appropriate definition of Δ and D, the expression is also valid, when the stability study includes several types of packages and storage conditions. That is, the formula presented herein is useful, both with and without allowance for differences between the different types of packages used for a specific pharmaceutical formulation.

Planning of Stability Studies

As a standard evaluation, the uncertainty of the slope is evaluated. The formula for that is D s in the terminology described above. The factor D is depending on the design. The factor s is independent of the design, but depends on which response is considered. As the total change over the shelf-life (ΔT) is the term in Allen's formula, we will for some expressions instead consider D T s.

The theoretically optimal use of a given number of determinations is to place half the observations at time 0 and the other half at the time point(s) when the calculations are made. That design is undesirable for several reasons; it is in conflict with the guidelines that request specific sampling times; it does not account for the fact that calculations are done both during the study and after collecting all data and it works only for an even total number of determinations. It is the theoretical optimal solution, both when one batch and when multiple batches are studied, typically just as long as the number of sampling points is a multiple of 2 times the number of batches. All designs will be compared with that design by evaluating the relative efficiency of the design compared to the theoretically optimal designs. The RE is the ratio of variances of the theoretically optimal design to that of the actual design studied. It has a direct interpretation on the number of determinations scale. For example, if a three-batch design with 39 determinations has a relative efficiency of 0.47, it should theoretically give the same precision as an optimal design with 0.47 times the 39 determinations of a three-batch design, equaling 18.33 determinations. In practice this means that the design studied has a precision similar to an optimal design with 6 determinations for each of the three batches, 18 in total. An alternative interpretation is that one can suggest an optimal design, which not only has similar precision but, in fact, is better than the design studied by using 8 determinations per batch, 24 in total, giving a savings of 38% (15/39) of the observations.

The disadvantage of that approach is that it does not address the level of precision necessary, and, secondly, it does not address the way the calculations will be performed later, that is, that specifications will be evaluated by means of Allen's formula. A consequence of this is that the release limits and several other quantities do not enter the formula and cannot therefore improve stability study planning.

The idea of calculating specifications at the planning stage is to take the Allen formula and insert the values to the extent possible. (Allen, Paul V. et al.: Determination of Release Limits: A General Methodology, PHARM. RES. 8:1210 (1991)). That means the random quantities and s are substituted by values corresponding to probability 0.95. That yields specifications such that there is a 95% probability that the calculated specifications will be better and a 5% probability that they will be worse. Thus, lack of knowledge of the results of the planned stability study is substituted by a safety margin evaluated based on statistical principles.

To be precise, the probabilities are considered separately for Δ and s, implying that the combined probability is not evaluated. These values, of course, depend on the design of the stability study. According to the instant invention, the other factors needed for Allen's formula, or consequences of these factors, are inserted according to the chosen design.

That is, Δ₀ and s₀ are chosen according to assumptions based on expectations. The values of RL, k, T, α and the length of the stability study are pre-determined. The stability study design determines D and df, from which t and F are found.

Change Term

For the change term ΔT, the value estimated for Δ consists of the assumed value plus the normal distribution with mean 0 and standard error D T s₀, where s₀ is the assumed value of the intermediate precision, and D a known factor that is derived for each design. To have 95% probability of obtaining lower results, we multiply by a factor u, the one-sided normal distribution fractile. For 95% probability, the value is u=1.65. Thus we substitute by (Δ₀+D u s₀) T.

Variability Term

The variability term in Allen's formula includes two terms, the uncertainty on the slope and the intermediate precision variation on the release determination.

The formula for the total variance is t s √(D²T²+1/k), where t is the one-sided α level fractile with the number of degrees of freedom df that will be obtained in the stability study and s the intermediate precision. As s is a random quantity, it will be substituted by its 95% probability value, which is of the form F s₀, where F is the square root of the χ²/f-distribution value with the degrees of freedom df as described above. This implies that the whole term will have the form t F so √(D²T²+1/k).

Combining the Terms

Summing all the terms above, gives a necessary difference of
Δ₀T+s₀{D u T+F t√(D²T²+1/k)}=Δ₀T+Q s₀,
where Q={D u T+F t √(D²T²+1/k)} depends on the design of the stability study and the external factors, but is independent of the assumed values. The lowest value of Q is u/k and is obtained when the stability study is infinitely large (D=0, F=1, df=∞).
Results from Analysis of Existing Data

In an alternate embodiment of the current invention it is useful to use a variation of Allen's formula. This altered formulation assumes that the intermediate precision is the same in the stability studies and in the future batches, whereas the standard formulations allow for different values. So the relation imposed is sΔ=D s. The advantage of this is that s can be moved outside of the square root sign. This makes it clearer that there are just two random terms to be determined in the stability study, Δ and s. Furthermore, it is easier to keep track of the degrees of freedom.

Assumptions involved in the current invention refer to the unknown parameters, that is, the rate of degradation Δ and the intermediate precision s. The aim of the stability studies is to determine these parameters. We may have some information on these values from earlier stability studies. Furthermore, release determinations and validation reports may give information on the intermediate precision. In the absence of such information, it may be possible to suggest values based on earlier formulations or on other, similar products. When it is important to discriminate between the assumed value and other values of a quantity, a subscript 0 will be used for the assumed value.

Change Over Time

The change over time is, of course, a very important factor, and a key quantity to be determined in the stability studies. The stability studies will, of course, not deliver the assumed value as a result; it will differ both due to random error and due to error in the assumed value. It is therefore, optimal to provide a design value rather than an assumed value. Providing this design value is preferably done in the initial stages to aid in subsequent calculations. With the provision of this design value the interpretation of any calculated results will result in improved accuracy and reliability. When the stability studies are performed the actual value, that is the value estimated based on collected data, makes much more sense than the assumed value. The assumed value may be “realistic” in the sense of being our best estimate, or it may be a worst-case suggestion, like the upper, or rather worst, confidence limit obtained in the previous experiments.

From a conceptual point of view, the assumed value will not be used in Allen's formula. In the present invention what is used is the stability study value, which reflects the true value plus random error. The true value will be substituted by the assumed value, and the random error will be accounted for by using a value corresponding to a 95% one-sided probability. The latter can be interpreted so as that we add a safety margin to account for the lack of knowledge on what will be the result of the stability study. The consequence is that mathematically, the assumed value enters additively.

An overview of some sources of information that can be used to suggest sensible values for the expected change over time is given in the table below. The various suggestions are listed in a preferred prioritized order.

TABLE 1
Some sources for suggesting values for the change over time

Previous long-term stability studies of the same drug in the same
formulation
Previous long-term stability studies of the same drug in other formulations
Previous accelerated stability studies of the same drug (temperature
corrected by Arrhenius formula)
Previous experience with similar drugs

Intermediate Precision Value

The intermediate precision will be estimated in the stability studies. However, as for previous quantities, we need a design value, or an assumed value. Inspiration as to which value to choose can be found, for example, in validation reports, results regarding other products, and/or earlier results. In the latter case, there is both a “realistic” value (“intermediate precision SD”) and a “worst case” value, the upper confidence limit for the intermediate precision.

An overview of some sources of information that can be used to suggest sensible values for the intermediate precision is given by Δ in the table below. The various suggestions are to some extent listed in preferred prioritized order. The choice, of course, also depends on the amount of information available for each potential source.

TABLE 2
Some sources for suggesting values for the intermediate precision

Previous long-term stability studies of the same drug in the same
formulation
Experience with the method of analysis, for example, quality
control samples
Validation reports for the method of analysis
Previous long-term stability studies of the same drug in other formulations
Previous accelerated stability studies of the same drug
Available release data of the same drug (if there are multiple
determinations for some batches)
Previous experience with similar drugs

Design Factors

Among the design factors, we include not only aspects that are directly related to the design of the stability study, like number of batches in the stability program, number and timing of samples, but also factors that refer to the frame within which the stability studies are run, like the time of evaluating the results. Finally, factors that are external to the stability studies are discussed here, the release limit and the length of shelf-life.

Instead of considering the length of the shelf-life, and calculating the shelf-life limit, one may choose the shelf-life limit and calculate the length of the shelf-life. Those two ways of considering the problem are not conflicting. In particular at the design stage, it is a matter of finding a design that will yield a satisfactory combination of shelf-life period and shelf-life specifications.

Release Specifications

The release specifications are preferably considered fixed at given values during most of this work. In practice, that may not strictly be the case, as the shelf-life limit may be set according to patient safety results for example. It should, however, be clear from the following how the release limits relate to the whole, so it should be simple to modify the release limits if necessary.

Length of Shelf-Life

The length of the shelf-life will be considered chosen beforehand during the calculations. In practice, this is a factor that may be modified, but then other suggestions for the length of the shelf-life can simply be inserted in the formulas.

In a following example, a shelf-life of 2 years has been used.

Shelf-Life Specifications

The present method cotemplates the development of satisfactory shelf-life specifications as the end result. If other design factors are desired as endpoints, one may try out several values of that design factor and pick the one with a satisfactory value for the shelf-life specifications.

As an alternative to choosing the length of the shelf-life, one may fix the specifications and then find the length in order to satisfy these specifications. Examples are when there are requirements from the authorities that the content should be above some standard limit, and/or when there is medical evidence that values outside given specifications have practical inconveniences.

Time of Evaluating Specifications—Length of Stability Studies

The length of the stability study is a critical factor for optimizing precision. Based on standard calculations (that is, the standard error principle), if a stability study is extended to double duration, only one quarter of the observations are necessary to give the same precision on the rate of degradation. In practice, there are two things that limit the length of stability studies. The first is that in extended studies the drug will cease to conform to reasonable specifications. The more positive side of such studies is that they can be extended over the current shelf-life, in order to examine whether the shelf-life period can be extended. The second point is the desire for quick information. Usually, there is pressure to complete the development phase as quickly as possible, making it important to decide on specifications as early as possible. So we need to decide when the specifications should be evaluated, using the information available at the given time as the criteria. Thus, when referring to the length of a stability study, we mean the effective length, that is, the length before making the specification calculations. That means that in practice, the stability studies continue, which makes it possible to update the specifications or extend the shelf-life period later, when more data are available.

Official requirements say that the company can only request a given shelf-life period, when the stability studies at the time of submission have a length at least half of the requested shelf-life when data are submitted. For making such an extrapolation, it is also required that the accelerated stability studies have given satisfactory results.

Number of Batches

Each batch yields information, so in that sense it is relevant to include as many batches as possible. On the other hand, including a batch also has a price in terms of resources. Furthermore, there is a practical upper limit set by the number of batches produced.

The authorities generally request that at least three batches are included. Three batches are used for the calculations in the various examples given herein, but other numbers may be used. The production process must be the final one, but it is not necessary to make full production scale batches.

The planning evaluations are based on all batches having the same slope (degradation rate), whereas they are allowed to start at different values.

Sampling Times

The most informative samples are those taken at the extreme time points, that is, the earliest one (time=0) and the last one, which in most cases should be interpreted as the last one before doing the calculations—alternatively it can be at the end of the current or desired shelf-life. In particular, the time=0 value is important for all possible choices of the time to make the evaluation, so this value must be determined well.

Official guidelines state sampling times of 0, 3, 6, 9 and 12 months during the first year. During the second year, sampling times of 18 and 24 months are used. Other values may be used as a matter of design choice.

Number of Repetitions

The number of replicates at each time point may be chosen, typically as 1, 2 or 3. Doing replicates in the same run is typically not beneficial as the day-to-day variation is important. In fact, when this specification herein discusses replications, it is always referring to the case of replications on different days.

Calculating Specifications at the Planning Stage

The idea of calculating specifications at the planning stage in accordance with the present invention is to take the Allen formula and insert the values to the extent possible. That means that the random quantities Δ and s are substituted by values corresponding to probability 0.95. That gives specifications such that there is 95% probability that the calculated specifications will be better and 5% probability that they will be worse. In common terms, lack of knowledge on the results of the planned stability study is substituted by a safety margin evaluated based on statistical principles.

To be precise, the probabilities are considered separately for Δ and s, implying that the combined probability is not evaluated. These values, of course, depend on the design of the stability study. The other factors are inserted according to the chosen design.

That is, Δ₀ and s₀ are chosen according to assumptions based on expectations. The values of RL, k, T, Δ and the length of the stability study are pre-determined, external to the stability study. The stability study design determines D and df, from which t and F are found.

Possible Designs

In order to examine the stability study design, we need to suggest assumed values for the parameters and evaluate the various possible designs.

Exemplary values of the external factors are
RL All results will be given as differences to RL, so that this parameter is not fixed.
k 1
T 2 years
α 95%
Stability study length 1 year (typically the time of submission)
Number of batches 3

Allen's formula operates with the limit in one direction being the important one, that is, in the direction of the expected change, with the other side placing less important restrictions on the batch. For the stability study planning, preferably only the important direction is considered and reported.

Overview of Designs Considered

The basic design used for comparisons provides for one determination at each sampling time point; that is, both initially and after 3, 6, 9 and 12 months of storage.

The other exemplary designs considered herein consist of multiple determinations at time 0 and 12 months, and only one sample for each batch at intermediate times (3, 6 and 9 months). The number of sample times at time 12 months varies from 1 to 8. The number of samples at time 0 is in principle the same, but due to the enormous importance of the initial time point, the number of samples at this time point is preferably at least 3, except for the first design. The first three columns give the number of samples at the various time points and the other columns give various helpful quantities for the whole design, that is, for three batches together.

TABLE 3
Overview designs considered. Three batches

| | Samples 3, | Samples | | | | | | |
|---|---|---|---|---|---|---|---|---|
| Samples | 6, 9 | 12 | | | | | | |
| initially | months | months | n | DT | df | F | t | Q |
| 1 | 1 | 1 | 15 | 1.461 | 11 | 1.337 | 1.796 | 6.655 |
| 3 | 1 | 1 | 21 | 1.165 | 17 | 1.274 | 1.740 | 5.320 |
| 3 | 1 | 2 | 24 | 0.996 | 20 | 1.253 | 1.725 | 4.690 |
| 3 | 1 | 3 | 27 | 0.906 | 23 | 1.237 | 1.714 | 4.351 |
| 4 | 1 | 4 | 33 | 0.792 | 29 | 1.211 | 1.700 | 3.930 |
| 5 | 1 | 5 | 39 | 0.713 | 35 | 1.193 | 1.690 | 3.648 |
| 6 | 1 | 6 | 45 | 0.653 | 41 | 1.178 | 1.683 | 3.444 |
| 7 | 1 | 7 | 51 | 0.606 | 47 | 1.167 | 1.678 | 3.288 |
| 8 | 1 | 8 | 57 | 0.569 | 53 | 1.157 | 1.674 | 3.165 |

The designs will be described according to the number of samples, as listed in the first three columns of Table 3. For example, the third design, that is, the one listed in the fourth row of the table is denoted 3-1-2.

The Standard Error Principle

Based on these designs, it is possible to derive the uncertainty on the slope. This is a standard known way of evaluating the uncertainty of a study design. Preferably we use the degradation during shelf-life; that is, aiming at the term ΔT. The formula for the standard error of this is D T s, where D T is found in the table above and s is the intermediate precision standard deviation. This is illustrated in Table 5, with various values for s.

TABLE 4
Uncertainty of slope (SE(slope)). Three batches.
The unit is SD-unit/2 years

| Design | SD 0.5 | SD 1.0 | SD 1.5 | SD 2.0 |
|---|---|---|---|---|
| 1-1-1 | 0.730 | 1.461 | 2.191 | 2.921 |
| 3-1-1 | 0.583 | 1.165 | 1.748 | 2.330 |
| 3-1-2 | 0.498 | 0.996 | 1.494 | 1.992 |
| 3-1-3 | 0.453 | 0.906 | 1.359 | 1.812 |
| 4-1-4 | 0.396 | 0.792 | 1.188 | 1.584 |
| 5-1-5 | 0.356 | 0.713 | 1.069 | 1.425 |
| 6-1-6 | 0.327 | 0.653 | 0.980 | 1.306 |
| 7-1-7 | 0.303 | 0.606 | 0.910 | 1.213 |
| 8-1-8 | 0.284 | 0.569 | 0.853 | 1.137 |

The Specification Principle

For evaluating the possible obtainable specifications, it is further necessary to include the expected degradation (change) in the expression. This implies that the table becomes three-dimensional and it is therefore split according to the value of the intermediate precision standard deviations. The values follow in the next four tables. More general values can be found by interpolation or by using the formula. The expected change needed is the change during the whole shelf-life (ΔT).

No units are given in the tables. In fact, any unit can be used, just as long as all numbers are expressed in the same unit.

TABLE 5
Obtainable specifications (SLL-RL). Three batches.
Intermediate precision SD 0.5

| Design | ΔT: 0.2 | ΔT: 0.5 | ΔT: 0.8 | ΔT: 1.0 |
|---|---|---|---|---|
| 1-1-1 | 3.53 | 3.83 | 4.13 | 4.33 |
| 3-1-1 | 2.86 | 3.16 | 3.46 | 3.66 |
| 3-1-2 | 2.55 | 2.85 | 3.15 | 3.35 |
| 3-1-3 | 2.38 | 2.68 | 2.98 | 3.18 |
| 4-1-4 | 2.16 | 2.46 | 2.76 | 2.96 |
| 5-1-5 | 2.02 | 2.32 | 2.62 | 2.82 |
| 6-1-6 | 1.92 | 2.22 | 2.52 | 2.72 |
| 7-1-7 | 1.84 | 2.14 | 2.44 | 2.64 |
| 8-1-8 | 1.78 | 2.08 | 2.38 | 2.58 |

TABLE 6
Obtainable specifications (SLL-RL). Three batches.
Intermediate precision SD 1.0

| Design | | ΔT: 0.2 | ΔT: 0.5 | ΔT: 0.8 | ΔT: 1.0 |
|---|---|---|---|---|---|
| | 1-1-1 | 6.86 | 7.16 | 7.46 | 7.66 |
| | 3-1-1 | 5.52 | 5.82 | 6.12 | 6.32 |
| | 3-1-2 | 4.89 | 5.19 | 5.49 | 5.69 |
| | 3-1-3 | 4.55 | 4.85 | 5.15 | 5.35 |
| | 4-1-4 | 4.13 | 4.43 | 4.73 | 4.93 |
| | 5-1-5 | 3.85 | 4.15 | 4.45 | 4.65 |
| | 6-1-6 | 3.64 | 3.94 | 4.24 | 4.44 |
| | 7-1-7 | 3.49 | 3.79 | 4.09 | 4.29 |
| | 8-1-8 | 3.36 | 3.66 | 3.96 | 4.16 |

TABLE 7
Obtainable specifications (SLL-RL). Three batches.
Intermediate precision SD 1.5

| Design | ΔT: 0.2 | ΔT: 0.5 | ΔT: 0.8 | ΔT: 1.0 |
|---|---|---|---|---|
| 1-1-1 | 10.18 | 10.48 | 10.78 | 10.98 |
| 3-1-1 | 8.18 | 8.48 | 8.78 | 8.98 |
| 3-1-2 | 7.24 | 7.54 | 7.84 | 8.04 |
| 3-1-3 | 6.73 | 7.03 | 7.33 | 7.53 |
| 4-1-4 | 6.09 | 6.39 | 6.69 | 6.89 |
| 5-1-5 | 5.67 | 5.97 | 6.27 | 6.47 |
| 6-1-6 | 5.37 | 5.67 | 5.97 | 6.17 |
| 7-1-7 | 5.13 | 5.43 | 5.73 | 5.93 |
| 8-1-8 | 4.95 | 5.25 | 5.55 | 5.75 |

TABLE 8
Obtainable specifications (SLL-RL). Three batches.
Intermediate precision SD 2.0

| Design | ΔT: 0.2 | ΔT: 0.5 | ΔT: 0.8 | ΔT: 1.0 |
|---|---|---|---|---|
| 1-1-1 | 13.51 | 13.81 | 14.11 | 14.31 |
| 3-1-1 | 10.84 | 11.14 | 11.44 | 11.64 |
| 3-1-2 | 9.58 | 9.88 | 10.18 | 10.38 |
| 3-1-3 | 8.90 | 9.20 | 9.50 | 9.70 |
| 4-1-4 | 8.06 | 8.36 | 8.66 | 8.86 |
| 5-1-5 | 7.50 | 7.80 | 8.10 | 8.30 |
| 6-1-6 | 7.09 | 7.39 | 7.69 | 7.89 |
| 7-1-7 | 6.78 | 7.08 | 7.38 | 7.58 |
| 8-1-8 | 6.53 | 6.83 | 7.13 | 7.33 |

### EXAMPLE 1

### Assays

For an example on how to use the tables, we will use the assay. First one must consider the measurement variation. If, for example, this is 0.5%, in accordance with a validation report, this implies that table 6 above should be used. Next, one must consider the expected degradation. This may be, for example, 0.8% during shelf-life. That implies that the column ΔT 0.8 should be used. Suppose the release interval is 98-102%. In that case, it is the lower limit 98% that creates a problem. For shelf-life, suppose that 95% is desired. This gives a difference between release and shelf-life of 3%. This number is used when going down the ΔT 0.8 column in the table. The first design that comes under 3 is 3-1-3, implying that a design with three samples on each batch at the initial and 12 month time points and single determinations at 3, 6 and 9 months is sufficient.

We can further evaluate that if, for example, the production department would like to extend the release limit to 97.5-102.5%, the difference RL-SLL is only 2.5 and in order to have 95% probability of being able to demonstrate that the product can keep the shelf-life limit of 95%, a stability study with 7 determinations at the extreme time points is necessary.

As a second example, consider a degradation product, with a release limit of 1.5%, an expected formation of the degradation product of 0.1% during shelf-life and an intermediate precision standard deviation of 0.2%. There is no intermediate precision entry of 0.2; but by using units of per thousand instead of percent, we find that the release limit is 15, the expected change is 1, and the intermediate precision is 2. With the 3-1-1 design, we can be reasonably sure to be able to suggest a necessary difference of 11.64/1000, which is rounded up to 12/1000. That yields a shelf-life limit of 27/1000 (15/1000+12/1000), that is, 2.7%. Instead using the 3-1-3 design, can make us reasonably sure to be able to suggest a necessary difference of 9.70/1000, which is rounded up to 10/1000. That yields a shelf-life limit of 25/1000, that is, 2.5%.

The expected change of 0.1% during all of shelf-life is unreasonably small and was used just in order to be among the values suggested in the tables. Suppose a more realistic change was 1.0% (10/1000). In the value 11.64 for the 3-1-1 design, 1.0 is the term corresponding to ΔT. This must be subtracted and the relevant term added. Thus the necessary difference is 11.64-1+10=20.64 (rounded up to 21). Thus, we can be reasonably sure to be able to suggest a shelf-life of 3.6% (found as 15/1000+21/1000).

### EXAMPLE 2

### Evaluation of Existing Data

As an example of an actual drug, the evaluation of existing data has led to the following values for the change over time (Δ) and intermediate precision (s). Three tablet strengths are to be considered, but will be handled separately. Four different packages styles—blister and three sizes of plastic containers, are to be used. Submission of an NDA is planned to take place after one year of storage. It is expected that a shelf-life of two years is reasonable at the time of submission. Although several combinations of storage temperature and humidity are going to be used, only the standard conditions are described in the example.

TABLE 9
Quantitative assumptions behind stability planning evaluations

| | | Assumed | |
|---|---|---|---|
| | Assumed change | intermediate | |
| Response | over 1 year | precision | Release limits |
| Assay | 1% | 1.5% | 95-105 |
| Impurities (sum) | 0.1% | 0.07 | 3 (low strength) |
| | | | 1.5 (1 and 2 mg) |
| Loss on drying, | 0.1 | 0.3 | 3 |
| Hardness | −7 | 5 | 90 |
| Disintegration | 0.5 | 0.7 | 30 |

Full Program

The starting point for the evaluation is a long-term stability study with one determination at each time point (0, 3, 6, 9, 12, 18, 24, 36, 48 and 60 months) consisting of storage conditions 25/60. Other storage conditions can include 30/70 and an accelerated program consisting of storage condition 40/75 with determinations after 3 and 6 months.

In practice, we preferably design a program for 25/60 (which will then also be used for 30/70) and an accelerated program to be used for 40/75. The designs considered will ignore the 40/75 storage condition. The full program will for each batch require one determination at time 0 and 4 for later times, in order to cover the four different package types. This gives, for 3 batches 51 determinations before submission and 111 determinations in total. In this design, 3 determinations are at time 0 and 108 after real storage. These numbers are for each response and only at the 25/60 storage condition.

TABLE 10
Potential specifications after 2 years of storage
at 25/60 - full program

| | Standard | Necessary | |
|---|---|---|---|
| | error on | difference | Shelf-life |
| Response (unit) | slope | (95% probability) | limits |
| Assay (% of target) | 0.68 | 8.3 | 86.7 |
| Impurities (sum) (%) | 0.032 | 0.49 | 3.5 (0.5 mg) |
| | | | 2.0 (1 and 2 mg) |
| Loss on drying (%), | 0.136 | 1.5 | 4.5 |
| Hardness | 2.27 | 35 | 55 |
| Disintegration | 0.32 | 3.9 | 34 |
| (minutes) | | | |

TABLE 11
Overview of different stability designs

| | | | Matrixing at 3, | | |
|---|---|---|---|---|---|
| | | Bracketing | 6, 9, 18, 24, 36 | No. of | Matrixing at |
| | No. of | (package | months | determinations | 48 months |
| Name of | determinations | type | (fraction | at 12 months | (fraction |
| design | at start | excluded) | included) | per batch | included) |
| Full | 1 per tablet | | | 1 (each | |
| | batch | | | package type) | |
| Bracketed | 1 per tablet | DUMA 60 | | 1 (each | |
| | batch | | | package type) | |
| Matrixed and | 1 per tablet | DUMA 60 | ⅓ | 1 (each | ⅔ |
| bracketed | batch | | | package type) | |
| Matrixed, | 1 per pack | DUMA 60 | ⅓ | 1 (each | ⅔ |
| bracketed and | type (3 per | | | package type) | |
| boosted | batch) | | | | |
| Matrixed, | 2 per pack | DUMA 60 | ⅓ | 2 (each | ⅔ |
| bracketed and | type (6 per | | | package type) | |
| extra boosted | batch) | | | | |

Matrixing, Bracketing and Boosting in Actual Pharmaceutical Assays

For the purposes of this invention it is preferred that time points deliver useful information. In particular, it is useful for special emphasis to be put on the first and last observations. It is clear what is meant by the first, that is, the initial, whereas the last changes over time. The most important is the one used for setting specifications. However, as there are multiple determinations at this time point (due to the different package types), it is particularly important to include extra information at time 0. This is called boosting herein. The consequence of performing triplicates on different days at time 0 will be considered herein. This modification is relevant for all the above mentioned designs and will be specifically evaluated for the matrixed and bracketed design described above. The stability study provided in Table 11 above corresponds to the sampling of pharmaceutical tablets after packaging instead of before, so that there is one determination for each package type in every batch instead of one determination for each batch. This gives, for 3 batches, 27 determinations before submission and 51 determinations in total. Of these 9 determinations are at time 0 and 42 after real storage. These numbers are for each response and only at the 25/60 storage condition. The various terms in the evaluations are d.f.=23, D T=0.906, F=1.237, Q=4.351, RE=0.722. This is a further improvement in efficiency compared to previous programs.

TABLE 12
Potential specifications after 2 years of storage
at 25/60 - matrix, bracketed and boosted

| | Standard | Necessary | |
|---|---|---|---|
| | error on | difference | Shelf-life |
| Response (unit) | slope | (95% probability) | limits |
| Assay (% of target) | 0.68 | 8.6 | 86.4 |
| Impurities (sum) | 0.032 | 0.51 | ≦3.6 (low |
| (%) | | | strength) |
| | | | ≦2.1 (medium and |
| | | | high strength) |
| Loss on drying (%), | 0.136 | 1.6 | ≦4.6 |
| Hardness | 2.26 | 36 | ≧54 |
| Disintegration | 0.32 | 4.1 | ≦35 |
| minutes) | | | |

Matrixing, Bracketing and Extra-Boosting in Actual Pharmaceutical Assays

For assays of standard pharmaceutical preparations, the standard error is somewhat high and therefore, extra boosting may be considered relevant. For purposes of the current invention and in an actual assay it is preferred to include twice as many observations at time 0 and 12 months in order to obtain better precision at the time of submission. This gives, for 3 batches 45 determinations before submission and 69 determinations in total. Of these 18 are at time 0 and 51 after real storage. These numbers are for each response and only at the 25/60 storage conditions. The various terms in the evaluations are df=41, DT=0.653, F=1.178, Q=3.444, and RE=0.833. This is a further improvement in efficiency compared to the previous programs. For an assay of this nature, the standard error of the slope is reduced to 0.49% per year, and the necessary difference to 7.2%. Thus, the shelf-life limit can be 87.8%.

The designs can then be evaluated according to the number of samples used at various sub studies.

Evaluation of Resources

The resources needed for performing the stability studies are one key element of this approach. On one hand the stability study must deliver the precision needed, but on the other hand, the resource use should be minimized. Here resources refer not only to money, but also manpower (used both for setting up and storing the samples as well as for laboratory analysis of the samples) and drug substance. For the designs considered here, the need for resources can be evaluated by the number of samples for analysis.

Assumed Values

The assumed values for Δ and s are important for the result, and therefore any choice of stability study design should be based on seriously chosen values. That is clearly the weakest point of the approach to choosing the size of the stability study. On the other hand, it is necessary to have some idea of the results in order to establish a sensible stability study plan. The consequence is that the calculated specifications in the design tables above should not be interpreted too strictly, or in other words, that the designs are suggestions rather than recommendations, and will vary as a function of design choice. It is worth spending some time choosing relevant values.

Extending the Stability Study

The approach presented in the current invention focuses on selecting variables for use in Allen's Formula so that all the terms needed to determine specification limits are provided. This data can then be evaluated for accuracy through the completion of an actual stability study. In reality the stability study is designed to run for the desired shelf-life period or the shelf-life period we have chosen, possibly with an extension after end of shelf-life. This means that in the end, better specifications or a longer shelf-life period may be obtainable.

Batch Variation in Slope

As described above, it is inherent in the Allen formula that the rate of degradation is the same for all batches. (Allen, Paul V. et al.: Determination of Release Limits: A General Methodology, PHARM. RES. 8:1210 (1991)). In practice, there may be random variation in this slope, for example, due to variation between batches of excipients. It is possible also to extend Allen's formula by including an extra random term describing this batch variation, but it will have a marked effect on the design of the stability study. Making evaluations during the planning stage requires determination of a further assumed value, namely the variation between batches. It requires more determinations and a large number of batches included.

Determinations at Time 0

It should be clear from the above that a good determination at time 0 is important. It is important because, it is the first and last determinations that are the most informative for evaluation of a slope. However, there are two additional advantages of determinations at time 0. The stability study is typically analyzed several times, at least after one year and after end of the study, but there could be further evaluations. What is meant by the “last” observation changes with the time of analysis, but the “first” observation is always the time 0 observation. Therefore, the initial determinations have a major importance for all the interim evaluations as well as the final evaluation of stability. Secondly, as there typically are also one or more accelerated storage conditions, these can be started simultaneously and thus the time 0 determination can be shared between the storage conditions. This implies that the cost of doing multiple observations at time 0 is small compared to the overall influence of this determination.

Time of Evaluating Specifications—Length of Stability Studies

The length of the stability study is one of the most critical factors for the obtainable precision. Based on standard calculations, if a stability study is extended to double duration, only one quarter of the observations is necessary to give the same precision on the rate of degradation. In practice, there are two things that limit the length of stability studies, one is that studies extending so long that the drug does not confirm to reasonable specifications do not make sense. (Wang, Wei, Instability, Stabilization, and Formulation of Liquid Protein Pharmaceuticals, INT'L J. OF PHARMACEUTICS, 185:129-88 (1999)). The more positive side of this is that studies can be extended over the current shelf-life, in order to examine whether the shelf-life period can be extended. The other point is the desire for quick information. Usually, there is time pressure during the development phase, making it important to decide on specifications as early as possible. So we need to decide when the specifications should be evaluated. What matters is the information available at that time point, so when we talk about the length of a stability study, we mean the effective length, that is, the length before making the specification calculations. That means that in practice, the stability studies continue, which makes it possible to update the specifications (or extend the shelf-life period) later, when more data are available.

Calculating Specifications at the Planning Stage

The idea of calculating specifications at the planning stage is to take the Allen formula and insert the values to the extent possible. That means the random quantities Δ and s are substituted by values corresponding to probability 0.95. That gives specifications so that there is 95% probability that the calculated specifications will be better than 5% probability that they will be worse. In common terms, lack of knowledge on the results of the planned stability study is substituted by a safety margin evaluated based on statistical principles.

To be precise, the probabilities are considered separately for Δ and s, implying that the combined probability is not evaluated. These values, of course, depend on the design of the stability study. The other factors are inserted according to the chosen design.

That is, Δ₀ and s₀ are chosen according to assumptions based on expectations. The values of RL, k, T, α and the length of stability study are predetermined external to the stability study. The stability study design determines D and df, from which t and F are found.

Long-Term Studies:

Typically, these are run at the intended storage conditions for the final commercial pharmaceutical product. For example, insulin is kept in refrigerated conditions (at 5 degrees Celsius). For tablets, this is 25 degrees Celsius and some standard humidity, for example 60% relative humidity. Some geographical regions operate with higher temperatures and/or humidity's. The length of these studies is the intended shelf-life (sometimes a little longer); but it is possible to file a drug application before the stability study is complete. The aim of these studies is to document that the drug quality is acceptable during the whole of shelf-life.

Accelerated Studies:

These studies are run at worse conditions than those expected. Such conditions may include higher temperatures, higher humidity, extra light or a vibrating environment. The aim of this part is two-fold: to document that the drug does not become unsafe during shorter periods of worse conditions, including the in-use period; and, to determine which degradation products develop so that they can be characterized and quantified in the long-term studies. Results of the accelerated studies must be acceptable in order to support extrapolation if the long-term studies are not run for the whole shelf-life length. An additional aim of these studies is to document that any restrictions put on the long-term studies are reasonable; for example, if accelerated studies of insulin at room temperature did not show degradation, there would be no basis for requesting storage in a refrigerator.

The design principles described in this document are most relevant for long-term studies, but the teachings herein may be applied to any study length as a matter of design choice. The design principles of the present invention concern the setting of specifications. That is particularly the case for NDA stability studies, which are done in order to set specifications to be used for the marketed product. Those specifications are set according to Allen's formula.

Extending the Stability Study

The approach presented here for planning a stability study applies to the stability study until the time of evaluating the specification. In reality the stability study is designed to run for the desired shelf-life period, possibly with an extension after end of shelf-life. This means that in the end, better specifications or a longer shelf-life period may be obtainable.

Robustness

Studies are generally designed in order to be able to let each product producer and each strength combination be considered separately. That allows the exclusion of one strength if its stability is not acceptable. Similarly, it makes it possible to submit the file for approval when data from the first producer is available. However, one preferably would analyze all available data jointly, in order to get the most precise results and in order to be able to compare the strengths and the producers.

That also means that if one type of package shows unacceptable stability, the data may still be sufficient, after combining the strengths. If one strength of formulation or one package type is excluded for reasons not related to stability, it may still be included in the stability evaluation, according to the guideline.

Using data from the available stability studies, various designs have been examined. For most responses a matrixed, bracketed and boosted design is recommended. For assay, however, this does not seem to deliver the desired precision and an extra boosting in accordance with the current invention is preferred.

Although the foregoing invention has been described in some detail by way of illustration and example for purposes of understanding, it will be apparent to those skilled in the art that certain changes and modifications may be practiced. Therefore, the description and examples should not be construed as limiting the scope of the invention, which is delineated by the appended claims.

Accordingly, it is to be understood that the embodiments of the invention herein providing for a more precise evaluation of pharmaceutical preparation methods and the precise determination of chemical stability achieved through modifying stability study methods are merely illustrative of the application of the principles of the invention. It will be evident from the foregoing description that changes in the form, methods of use, and applications of the elements of the disclosed stability study methodology and resulting pharmaceutical compositions may be resorted to without departing from the spirit of the invention, or the scope of the appended claims.

1. A method for planning a stability study of a pharmaceutical composition comprising: a) selecting a value for a release limit variable for a given specification test; b) selecting a desired length of the shelf-life of said pharmaceutical composition; c) selecting a time at which an analysis of the data for said stability study will be performed; d) selecting time points at which one or more measurements of one or more predetermined pharmaceutical test variables will be performed; e) selecting a number of measurements of said predetermined test variables that will be performed at each of said time points; f) selecting a value for an expected degradation rate of said pharmaceutical composition over time; g) selecting a value for an intermediate precision of said measurements; h) selecting a probability level regarding the level of certainty of the outcome of said stability study, and i) preparing at least one batch of said pharmaceutical composition for use in said stability study based upon the variables selected in steps a) through h).

2. The method of claim 1 wherein the selected value of said expected degradation rate is based on previous long-term stability studies.

3. The method of claim 1, further comprising calculating the shelf-life specification limits of said pharmaceutical composition based upon the variables selected in steps a) through h).

4. The method of claim 3 further comprising optimizing the variables selected in steps a) through h) by changing one or more of said variables as a function of said calculation.

5. The method of claim 3 wherein the specification test limits are re-calculated by substituting in actual data obtained during said stability study for one or more of the variables selected in steps a) through h).

6. The method of claim 5 further comprising optimizing the variables selected in steps a) through h) by changing one or more of said variables as a function of said calculation.

7. The method of claim 6 wherein said probability level regarding the level of certainty is at least 95%.

8. The method of claim 1, wherein said probability level is at least 90%.

9. The method of claim 1 wherein said probability level is 95%.

10. The method of claim 1 wherein the selected value of said expected degradation rate is based on previous long-term stability studies of said pharmaceutical composition in alternate formulations.

11. The method of claim 1 wherein the selected value of said expected degradation rate is based on previous accelerated stability studies of said pharmaceutical composition.

12. The method of claim 11 wherein the selected value is based on accelerated stability results that are temperature corrected by the Arrhenius formula.

13. The method of claim 1 wherein the selected value of said intermediate precision of the analysis of said pharmaceutical composition is determined from previous long-term stability studies of said pharmaceutical composition.

14. The method of claim 1 wherein the selected value of said intermediate precision of the analysis of said pharmaceutical composition is determined from previous accelerated stability studies of said pharmaceutical composition.

15. The method of claim 1 wherein the selected value of said expected degradation rate is based on previous long-term stability studies of said pharmaceutical composition while the selected value of said intermediate precision of the analysis of said pharmaceutical composition is determined from conducting a stability study of said pharmaceutical composition.

16. The method of claim 1 wherein the selected value of said expected degradation rate is based on conducting a stability study of said pharmaceutical composition while the selected value of said intermediate precision of the analysis of said pharmaceutical composition is determined from previous long-term stability studies of said pharmaceutical composition.

17. The method of claim 1 wherein the time points for measurement of the variables selected in steps a) through h) are at 0, 3, 6, 9, and 12 months after start of the stability study of said pharmaceutical composition.

18. The method of claim 1 wherein the shelf-life specification limits of said pharmaceutical composition is calculated utilizing the Allen Formula.

19. The method of claim 1 wherein the shelf-life specification limits of said pharmaceutical composition are calculated utilizing the Allen Formula such that the probability level of said pharmaceutical composition satisfying its specification tests is at least 95%.

20. The method of claim 1 wherein said pharmaceutical composition is administered through an oral administration of a pharmaceutical formulation.

21. (canceled)

22. The method of claim 20 wherein the packaging for said pharmaceutical formulation varies.

23. The method of claim 20 wherein the dosage strength of an active ingredient in said pharmaceutical formulation varies.

24. A method of determining shelf-life specifications of pharmaceutical composition, comprising: a) selecting a value for a release limit variable for a given specification test; b) selecting a desired length of the shelf-life of said pharmaceutical composition; c) selecting a time at which an interim analysis will be performed; d) selecting time points at which one or more measurements of one or more predetermined pharmaceutical test variables will be performed; e) selecting a number of measurements of said predetermined test variables that will be performed at each of said time points; f) selecting a value for an expected degradation rate of said pharmaceutical composition over time; g) selecting a value for an intermediate precision of said measurements; and h) selecting a probability level regarding the level of certainty of the outcome of said stability study; i) calculating the shelf-life specification limits of said pharmaceutical composition based upon the variables selected in steps a) through h), and j) preparing at least one batch of said pharmaceutical composition based upon the shelf-life specifications calculated in step i).

25-44. (canceled)

45. A method for planning and conducting a stability study of a pharmaceutical composition comprising: a) selecting a value for a release limit variable for a given specification test; b) selecting a desired length of the shelf-life of said pharmaceutical composition; c) selecting a time at which an interim analysis will be performed; d) selecting time points at which one or more measurements of one or more predetermined pharmaceutical test variables will be performed; e) selecting a number of measurements of said predetermined test variables that will be performed at each of said time points; f) selecting a value for an expected degradation rate of said pharmaceutical composition over time; g) selecting a value for an intermediate precision of said measurements; and h) selecting a probability level regarding the level of certainty of the outcome of said stability study; i) calculating the shelf-life specification limits of said pharmaceutical composition based upon the variables selected in steps a) through h); j) optimizing the variables selected in steps a) through h) by changing one or more of said variables as a function of said calculation; and k) conducting said stability study for said pharmaceutical composition based on said optimized values selected for said pharmaceutical composition.

46-63. (canceled)

64. The method of claim 1 wherein in said analysis is an interim analysis.

65. The method of claim 64 wherein in said interim analysis is performed at least once.

66. The method of claim 1 further comprising selecting the number of batches of said pharmaceutical composition to be prepared is determined.

67. The method of claim 1, wherein at least one batch of said pharmaceutical composition is prepared.

68. The method of claim 67, wherein at least three batches of said pharmaceutical composition are prepared for testing in said stability study.

69. The method of claim 68 wherein said at least one batch of said pharmaceutical composition is tested for degradation.

70-81. (canceled)
